(19) United States
(12) Patent Application Publication
(10) Pub. No.: US 2004/0044327 A1
(43) Pub. Date: Mar. 4, 2004

(54) Transfer needle assembly

(76) Inventor: Mitsuru Hasegawa, Osaka-shi (JP)

Correspondence Address: KUBOVCIK & KUBOVCIK, SUITE 710, 900 17TH STREET NW, WASHINGTON, DC, 20006

(21) Appl. No.: 10/652,512

(22) Filed: Sep. 2, 2003

(30) Foreign Application Priority Data
Sep. 4, 2002 (JP) 2002-259227

(51) Int. Cl. A61B 19/00

(52) U.S. Cl. 604/414; 604/411

(57) ABSTRACT

A transfer needle assembly includes a tubular guide member 1 having a top surface 11, a skirt portion 13, and a puncture needle insertion portion 12; and a puncture member 2 of which tip 223 is fitted in the puncture needle insertion portion 12 such that the tip 223 can slide from a position where it is receded into the skirt portion 13 to a position where it projects from the skirt portion 13. The puncture member 2 is a tubular member having a tip fitting portion 21 at the proximal end thereof and, at the distal end thereof, a puncture needle 22 to be inserted into the puncture needle insertion portion 12. The puncture needle 22 is provided with a liquid passage 222 that communicates with the tip fitting portion 21 and a gas passage 221 that is open to the outside through an air filter 231.

### FIELD OF THE INVENTION

The present invention relates to a transfer needle assembly for transferring fluid from a fluid source to a vial containing a solid material or a liquid material. The transfer needle assembly of the present invention eliminates problems such as contamination of a tip of a puncture needle in the transfer needle assembly before use and injury to an operator's finger by the tip of the puncture needle and fluid leakage from the transfer needle assembly when in use.

### BACKGROUND OF THE INVENTION

In order to prepare a liquid medical component for adhering tissues in a living body or to be mixed with a solid component at a medical facility, or to be injected by a patient himself for home medical treatment, a dried or liquid medicine contained in a vial is dissolved with a fluid such as saline to make a medicinal solution which is then transferred to a syringe. A syringe with a puncture needle or a tool having two hollow puncture needles at both ends, called a “double-ended needle”, has been used for these operations. When the syringe with a needle is used, a rubber stopper in a mouth portion of a fluid container is pricked with the puncture needle to collect a predetermined amount of the fluid into the syringe and then, a rubber stopper in a mouth portion of a vial is pricked with the same needle to inject the fluid into the vial from the syringe. Then, the vial is shaken to dissolve the medicine with the fluid and the rubber stopper is pricked with the above needle again to collect a predetermined amount of the medicinal solution into the syringe. When the “double-ended needle” is used, the rubber stopper of the vial filled with a dried medicine is pricked with one puncture needle of the double-ended needle and the rubber stopper of the fluid container is pricked with another puncture needle, respectively, which are located at both ends of the double-ended needle to communicate the vial with the fluid container. The fluid in the fluid container is transferred to the vial to dissolve the dried medicine, then, the fluid container and the double-ended needle are removed from the vial, and finally, the rubber stopper of the vial is pricked with a syringe with a needle likewise to collect a predetermined amount of the medicinal solution into the syringe.

These prior techniques involve a problem in that when the procedure of dissolving a dried medicine is carried out using the syringe with a needle, the procedure is complicated, the tip of the puncture needle may be contaminated before use because the needle is exposed, and an operator may get injured by the tip of the needle when in use. When the double-ended needle is used and the needle is pulled out or a predetermined amount of the medicinal solution is collected into the syringe, the operator may get injured by the tips of the needle, and the tips of the needle may be contaminated before the collection of the medicinal solution because the tips of the needle are exposed like the syringe with a needle. To solve the above problems, there is proposed an admixture injection adaptor which enables both injection and collection of a medicinal solution during a dissolution procedure (refer to JP 7-213585 A, paragraphs [0008] to [0012], FIG. 1 and FIG. 2)

In the admixture injection adaptor in this reference, a hollow puncture needle and a tubular tip fitting part, respectively, are set on opposite sides on the axis of a discoid hub, and a tubular vial fitting part which is concentrically extended over the tip of the puncture needle and a tubular syringe fitting part which is concentrically extended over the tip of the tip fitting part are constructed on the outer edge of the hub. The adaptor can conveniently transfer a medicinal solution in a vial to a syringe after medicine in the vial is dissolved with a fluid to prepare the medicinal solution while contamination of the tip of the needle or injury by the needle is avoided. However, the length of the skirt is limited so as to prevent the end of the skirt from contacting with the shoulder of the vial when the vial is pricked with the needle, whereby the vial cannot be fitted straight and, therefore, a gap is formed between the rubber stopper of the vial and the needle to cause leakage of the medicinal solution during dissolution.

### SUMMARY OF THE INVENTION

The present invention has been made in view of the above circumstances, and it is, therefore, an object of the present invention to provide a transfer needle assembly having the following advantages: the dried medicine can be easily dissolved; contamination of the tip of the needle and injury of an operator by the tip of the needle can be avoided; and leakage of a fluid from the transfer needle assembly during dissolution can be prevented.

In order to solve the above problems, the inventor of the present invention has carried out extensive studies. The inventor understood that it would be desirable that the tip of the puncture member be capable of sliding from a position where the tip is receded in a skirt portion to a position where the tip projects from the skirt portion during transferring and injecting of a fluid, thereby reaching the present invention. Namely, the present invention relates to a transfer needle assembly comprising;

a cap-like guide member 1 having a top surface 11, a skirt portion 13 and a tubular puncture needle insertion portion 12 penetrated through the top surface 11 in the axial direction of the cap-like guide member 1; and

a puncture member 2 comprising a tubular member provided with a tip fitting portion 21 at the proximal end thereof for fitting a tip of a syringe and a puncture needle 22 at the distal end thereof to be inserted into the puncture needle insertion portion 12 of the cap-like guide member 1, and a filter portion 23;

wherein the puncture needle 22 has a needle tip 223 and is provided with a gas passage 221 and a liquid passage 222; and the liquid passage 222 communicates with the tip fitting portion 21 and the gas passage 221 is open to the outside through an air filter 231 in the filter portion 23, and

wherein the puncture member 2 is fitted in the puncture needle insertion portion 12 of the cap-like guide member 1 in such a manner that the needle tip 223 of the puncture needle 22 in the puncture member 2 slides from a position where the needle tip 223 is receded in the skirt portion 13 to a position where the needle tip 223 projects from the skirt portion 13.

Here, it is desirable that a cutout 14 is formed in the skirt portion 13 of the guide member 1 so that the puncture needle 22 which recedes into the skirt portion 13 can be seen. A disk-like, preferably sector disk-like collar 24 may also be provided at the proximal end of the puncture needle 22 to stop sliding in a forward direction of the puncture member 2 when the collar 24 comes in contact with the proximal end 122 of the puncture needle insertion portion 12. It is also desirable that the inner wall of the puncture needle insertion portion 12 is undercut to form a stepped portion 121 while retaining the proximal end portion thereof, an intermediate portion of the puncture needle 22 is provided with an annular projection 224, and the stepped portion 121 formed by undercutting engages with the annular projection 224 to stop sliding of the puncture member 2 in a backward direction.

Slide suppression means 32 for preventing accidental sliding of the puncture member 2 may also be provided between the collar 24 of the puncture member 2 and the top surface 11 of the guide member 1. The slide suppression means is suitably a spring or a prop.

### BRIEF DESCRIPTION OF THE DRAWINGS

FIG. 1 is a front view of an embodiment of the present invention.

FIG. 2 is a sectional view cut on line X-X of FIG. 1.

FIG. 3 is a longitudinal sectional view of another embodiment of the present invention.

FIG. 4 is a front view of still another embodiment of the present invention.

FIG. 5 is a front sectional view of FIG. 4.

FIG. 6 is a sectional view cut on line Y-Y of FIG. 4.

FIG. 7 is a diagram for explaining use of the transfer needle assembly of the present invention.

FIG. 8 is a diagram for explaining use of the transfer needle assembly of the present invention.

### THE MOST PREFERRED EMBODIMENT OF THE INVENTION

Preferred embodiments of the present invention will be described with reference to the accompanying drawings.

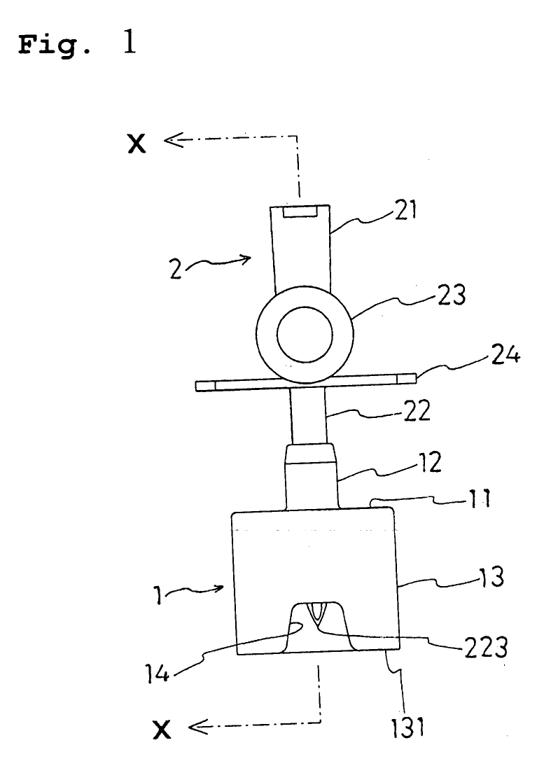

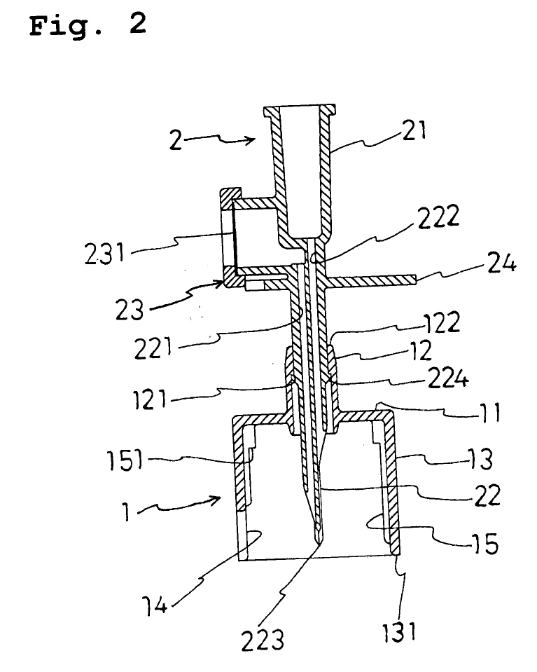

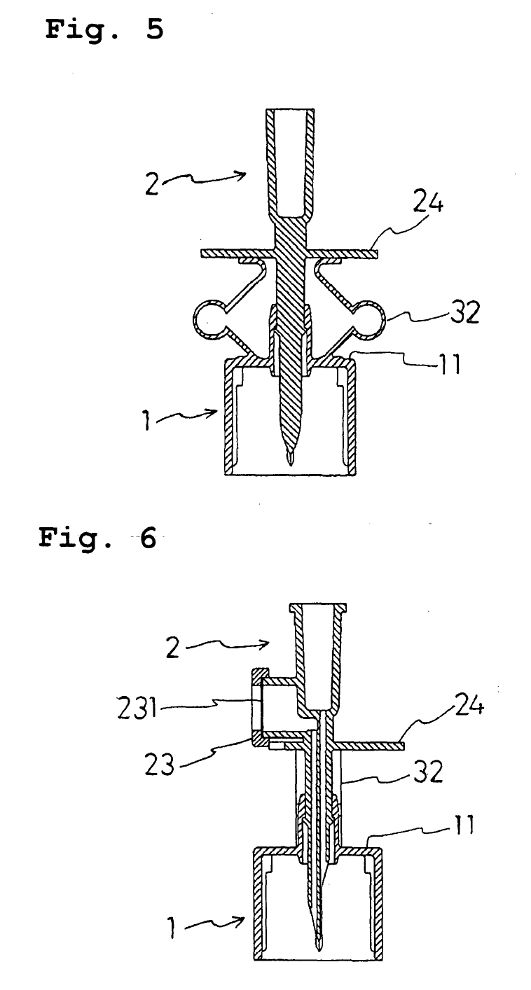

As shown in FIGS. 1 to 6, the transfer needle assembly of the present invention includes a cap-like guide member 1 having a top surface 11, a skirt portion 13 and a puncture needle insertion portion 12; and a puncture member 2 fitted in the puncture needle insertion portion 12 of the cap-like guide member 1 in such a manner that a needle tip 223 of a puncture needle 22 in the puncture member 2 slides from a position where the needle tip 223 is receded into the skirt portion 13 to a position where the needle tip 223 projects from the skirt portion 13.

The guide member 1 is generally a cap-like member formed from a plastic such as polypropylene, polyethylene, polyester, polyvinyl chloride, polycarbonate or ABS resin and has the top surface 11, the skirt portion 13 and the tubular insertion portion 12 for puncture needle 22 penetrated through the top surface 11 in the axial direction of the transfer needle assembly. The skirt portion 13 has an inner diameter that enables a mouth portion of a vial (not shown) to slide along the inner wall thereof and a cutout 14, formed on the distal end side thereof, for identifying the puncture needle 22 situated at a position where the needle is receded into the skirt portion 13. A plurality of (generally 3 to 5) vertical ribs 15 which enable the mouth portion of the vial to slide smoothly are provided on the inner wall of the skirt portion 13. The vertical ribs 15 are undercut with their proximal end portions retained adjacent to the top surface 11, and stepped portions 151 formed by the undercutting come in contact with the mouth portion of the vial when the end 131 of the skirt portion 13 advances to a position where the end 131 contacts the shoulder portion of the vial.

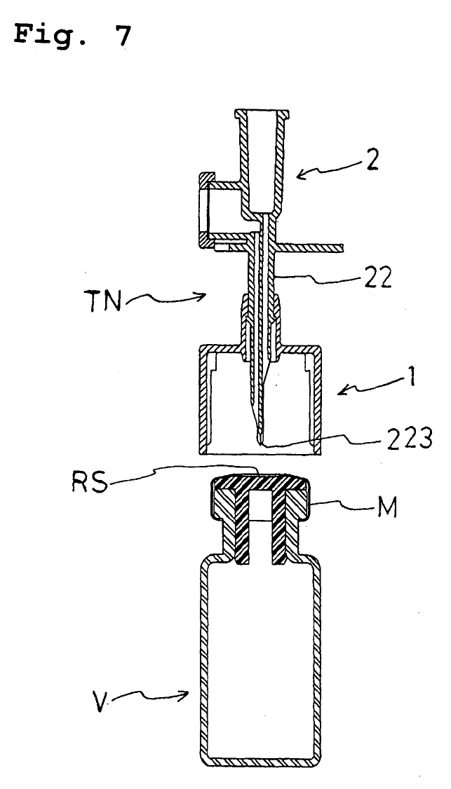

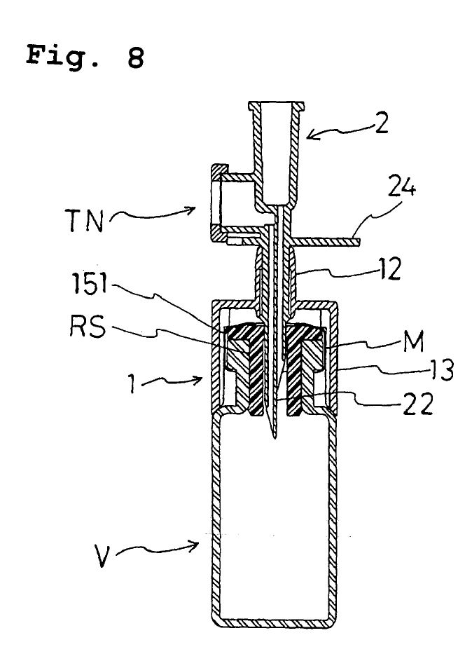

The inner wall of the puncture needle insertion portion 12 is undercut with part of its proximal end retained and the puncture needle 22 of the puncture member 2 is inserted into this portion in such a manner that the puncture needle 22 can slide along the inner wall. Owing to this structure, the puncture member 2 can recede up to a position where an annular rib 224 of the puncture needle 22 to be described hereinafter is engaged with a stepped portion 121 formed by the undercutting. Therefore, the undercutting position and the position of the annular rib 224 are determined such that the tip 223 of the puncture member 2, that is, the tip 223 of the puncture needle 22, is receded into the skirt portion 13 when the annular rib 224 and the stepped portion 121 engage with each other (see, FIG. 7). The puncture member 2 can advance up to a position where the proximal end 122 of the puncture needle insertion portion 12 comes in contact with a collar 24 of the puncture member 2 to be described hereinafter. Therefore, the projecting length of the puncture needle insertion portion 12 from the top surface 11 is determined such that the tip 223 of the puncture member 2 projects from the skirt portion 13 when the proximal end 122 of the puncture needle insertion portion 12 comes in contact with the collar 24 of the puncture member 2 (see, FIG. 8).

The puncture member 2 is generally a tubular member formed from a plastic such as polypropylene, polyethylene, polyester, polycarbonate or ABS resin and a tip 223 of the puncture needle 22 is fitted in the puncture needle insertion portion 12 of the guide member 1 in such a manner that the tip 223 can slide from a position where the tip 223 is receded in the inside of the skirt portion 13 to a position where the tip 223 projects from the skirt portion 13. The puncture member 2, has, at the proximal end thereof, the tip fitting portion 21 into which a tip (not shown) of a syringe can be mounted and, at the distal end thereof, the puncture needle 22 to be inserted into the puncture needle insertion portion 12.

In detail, the puncture member 2 is a tubular member having, at its proximal end, a tip fitting portion 21 and, at its distal end, a puncture needle 22 to be inserted into the above puncture needle insertion portion 12 and which also has a filter portion 23. The tip fitting portion 21 comprises a cylindrical area for accommodating a syringe and for mounting a tip of the syringe and an aperture on the bottom. The aperture is preferably, crescent, semicircular, elliptical or circular, but is not limited to these shapes. The puncture needle 22 includes a tubular outer wall of which a proximal end is partly connected with the bottom of the tip fitting portion 21, but the remaining proximal part is connected with the filter portion 23, and a needle shaped septum continued from the aperture of the tip fitting portion 21. The outer wall and the septum form a liquid passage from the tip fitting portion 21 to the guide member 1 and a gas passage from the filter portion 23 to the guide member 1. The cross-sectional area of the liquid passage is preferably, crescent, semicircular, elliptical or circular, and the cross-sectional area of the gas passage is, preferably, circular.

The length of the septum is longer than the length of the outer wall of the puncture needle, and the distal end of the outer wall of the gas passage extends downwardly beyond the distal end of the outer wall of the liquid passage in order to prevent the air introduced into the vial from returning to the syringe.

The filter portion 23 is located outside of the tip fitting portion 12 and connected with the outer wall of the puncture needle 22 and is provided with an air filter 231 at the proximal end of the gas passage. The filter portion 23 is preferably located in a position where a lock-type syringe does not contact the tip fitting portion 21 and further, a position where the movements of the disk-like collar 24 and the puncture needle fitting portion 12 are not disturbed when the collar 24 comes in contact with the puncture needle fitting portion 12. The filter portion 23 is, preferably, a cylindrical portion extending from the outside of the tip fitting portion 21 and the outer wall of the puncture needle 22, but is not limited to this shape.

That is, the puncture needle 22 is provided with a liquid passage 222 which communicates with the tip fitting portion 21 and a gas passage 221 which communicates with the outside through an air filter 231 in the filter portion 23.

The gas passage 221 enables outside air to be introduced into the mouth portion of the vial via the air filter 231 in the filter portion 23 and the liquid passage 222 enables a fluid to be injected into the mouth of the vial through the tip 223. The liquid passage 222 communicates with the tip fitting portion 21, and the gas passage 221 is open to the outside through the air filter 231 of the filter portion 23 which communicates with the gas passage 221.

The vial filled with a medicine such as a biological agent usually has a reduced pressure on the inside while freeze-drying the medicine. When a syringe filled with a dissolution fluid such as saline is connected with the vial by means of the transfer needle assembly, outside air is introduced into the vial in order to release the reduced pressure. The filter portion 23 is provided in the gas passage 221 to prevent air contamination via the air filter 231. In case of a syringe filled with a dissolution fluid and connected with the tip fitting portion 21, air is also introduced into the vial when the dissolution fluid is introduced into the vial containing a dried medicine and mixed to prepare the medicinal solution, and the solution is returned back into the syringe again.

In the present invention, for example, a disk-like, preferably, sector disk-like, collar 24 may be provided at the proximal end of the puncture needle 22 so that the collar 24 comes in contact with the proximal end 122 of the puncture needle insertion portion 12 at a position where the tip 223 of the puncture member 2 projects from the skirt portion 13 to stop the sliding of puncture member 2 in a forward direction.

The intermediate portion of the puncture needle 22 is provided with the annular rib 224 which is engaged with the stepped portion 121 of the puncture needle insertion portion 12 at a position where the tip 223 of the puncture member 2 is receded into the skirt portion 13 to stop the sliding of the puncture member 2 in a backward direction.

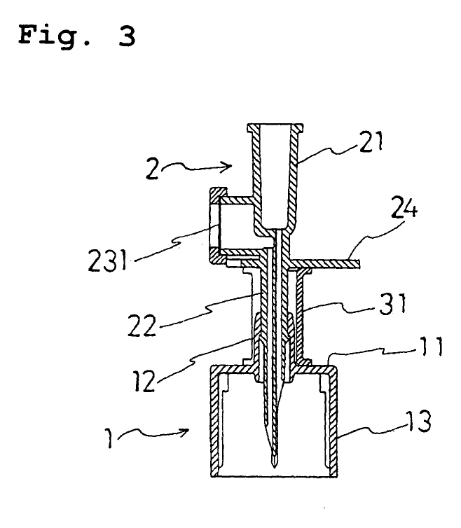

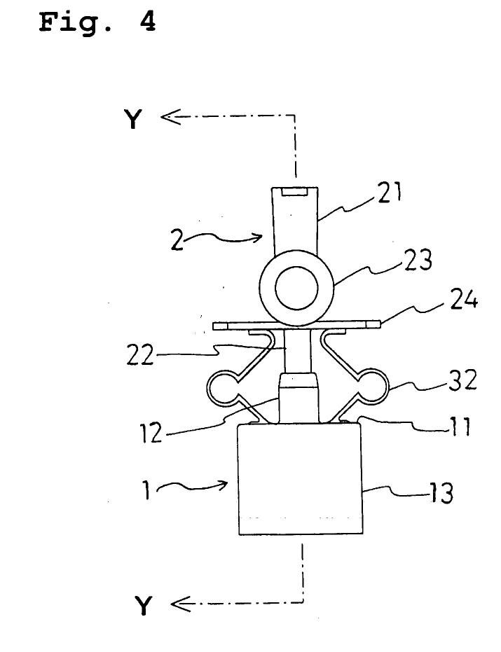

To prevent the puncture member 2 from sliding accidentally, slide suppressing means may be provided between the collar 24 of the puncture member 2 and the top surface 11 of the guide member 1. The slide suppressing means is suitably a spring 32 as shown in FIGS. 4 to 6 or a prop 31 having a corset function as shown in FIG. 3.

A description is given below of use of the transfer needle assembly of the present invention with reference to FIG. 7 and FIG. 8.

As shown in FIG. 7, a transfer needle assembly TN and a vial V are first prepared. The tip 223 of the puncture needle 22 is situated within the skirt portion 13. The skirt portion 13 is applied to the mouth portion M of the vial V, the whole transfer needle assembly TN is pressed down straight, a rubber stopper RS is pricked with the puncture needle 22, and the forward movement of the transfer needle assembly TN is stopped at a position where the puncture needle insertion portion 12 comes in contact with the sector disk-like collar 24. At this point, the stepped portion 151 engages the mouth portion M of the vial V, and the inside of the vial V communicates with the outside air and the tip fitting portion 21 through the gas passage 221 and the liquid passage 222 of the puncture needle 22, respectively (see FIG. 8). When the tip fitting portion 21 is connected to a syringe (not shown) filled with the fluid in this state, the dissolution procedure can be easily carried out by introducing the fluid in the syringe into the mouth portion M of the vial V.

As obvious from the above description, by using the present invention, the dissolution procedure can be easily carried out. Contamination of the tip of the needle and injury by the tip of the needle can be avoided and leakage of a liquid during the dissolution procedure can be prevented.

What is claimed is:

1. A transfer needle assembly comprising
a cap-like guide member 1 having a top surface 11, a skirt portion 13 and a tubular puncture needle insertion portion 12 penetrated through the top surface 11 in an axial direction of the cap-like guide member 1; and
a puncture member 2 comprising a tubular member provided with a tip fitting portion 21 at a proximal end thereof for fitting a tip of a syringe and a puncture needle 22 at a distal end thereof to be inserted into the puncture needle insertion portion 12 of the cap-like guide member 1, and a filter portion 23;
wherein the puncture needle 22 has a needle tip 223 and is provided with a gas passage 221 and a liquid passage 222; and the liquid passage 222 communicates with the tip fitting portion 21 and the gas passage 221 is open to the outside through an air filter 231 in the filter portion 23, and
wherein the puncture member 2 is fitted in the puncture needle insertion portion 12 of the cap-like guide member 1 in such a manner that the needle tip 223 of the puncture needle 22 in the puncture member 2 slides from a position where the needle tip 223 is receded into the skirt portion 13 to a position where the needle tip 223 projects from the skirt portion 13.

2. The transfer needle assembly according to claim 1, wherein a cutout 14 is formed in the skirt portion 13 of the cap-like guide member 1 so that the needle tip 223 of the puncture needle 22 which is receded into the skirt portion 13 can be seen.

3. The transfer needle assembly according to claim 1, wherein a disk-like collar 24 is provided at the proximal end of the puncture needle 22 to stop sliding of the puncture member 2 in a forward direction when the collar 24 comes in contact with the proximal end 122 of the puncture needle insertion portion 12.

4. The transfer needle assembly according to claims 1, wherein an inner wall of the puncture needle insertion portion 12 is undercut to form a stepped portion 121 at a proximal end thereof, an intermediate portion of the puncture needle 22 is provided with an annular projection 224, and the stepped portion 121 formed by undercutting is engageable with the annular projection 224 to stop sliding of the puncture member 2 in a backward direction.

5. The transfer needle assembly according to claim 1, wherein a disk-like collar 24 is provided at the proximal end of the puncture needle 22 to stop sliding in a forward direction of the puncture member 2 when the collar 24 comes in contact with the proximal end 122 of the puncture needle insertion portion 12,
and wherein an inner wall of the puncture needle insertion portion 12 is undercut to form a stepped portion 121 at a proximal end thereof, an intermediate portion of the puncture needle 22 is provided with an annular projection 224, and the stepped portion 121 formed by undercutting is engageable with the annular projection 224 to stop sliding of the puncture member 2 in a backward direction.

6. The transfer needle assembly according to claim 1, wherein a slide suppression means 32 for preventing accidental sliding of the puncture member 2 is provided between the collar 24 of the puncture member 2 and the top surface 11 of the guide member 1.

7. The transfer needle assembly according to claim 6, wherein the slide suppression means 32 is a spring.

8. The transfer needle assembly according to claim 6, wherein the slide suppression means 32 is a prop.
